(19) United States
(12) Patent Application Publication
(10) Pub. No.: US 2023/0390519 A1
(43) Pub. Date: Dec. 7, 2023

(54) SYSTEM AND METHOD FOR PLANNING AND/OR CONTROLLING ENVIRONMENTAL FEATURES IN A SPACE FOR AFFECTING PHYSIOLOGICAL RESPONSE

(71) Applicant: Liat MAROM, Tel Aviv (IL)

(72) Inventor: Liat MAROM, Tel Aviv (IL)

(21) Appl. No.: 18/037,058

(22) PCT Filed: Nov. 16, 2021
(86) PCT No.: PCT/IL2021/051358

§ 371 Date: May 15, 2023

(30) Foreign Application Priority Data
Nov. 16, 2020 (IL) 278748

(51) Int. Cl. A61M 21/00; A61M 21/02

(52) U.S. Cl. CPC A61M 21/0094; A61M 21/02; A61M 2205/3303; A61M 2021/0016; A61M 2021/0027; A61M 2021/0022; A61M 2021/0044; A61M 2230/10

(57) ABSTRACT

The present disclosure provides a system and a method for affecting, planning and/or designing a space to result in a stimulation of one or more senses of a subject that is located in the space for inducing a desired physiological response to the subject. For example, by applying the output of the system or the method on the space, the space can induce a desired conditional, behavioral, and/or emotional response such as concentration, aggression, calmness, amazement, confusion, etc. The physiological response is manifested by a measurable physiological effect. For example, the physiological response can be exhibited by any one of a neuronal response such as the creation of a certain brainwave, hormone secretion, change in heart rate, respiration rate or blood pressure, or any combination thereof. In response to an input of a desired physiological response, environmental features are selected to be applied in the space to obtain the desired effect. The output data includes execution profile that may affect on any one of the senses of the subject.

### TECHNOLOGICAL FIELD

The present disclosure is in the field of space designing, in particular space designing for affecting physiological response in the space.

### BACKGROUND ART

References considered to be relevant as background to the presently disclosed subject matter are listed below:
U.S. Ser. No. 10/599,116
US 2019/0209806

Acknowledgement of the above references herein is not to be inferred as meaning that these are in any way relevant to the patentability of the presently disclosed subject matter.

### BACKGROUND

Indoor and outdoor spaces are sometimes intended to serve for a certain functionality. This functionality may involve a physiological or emotional response of people that present in the space or carrying out a certain activity in the space. The physiological and/or emotional response may contribute or interrupt the functionality of the space and therefore it is desired to control this physiological response to ensure that the functionality and the purpose of the space is maximized.

### GENERAL DESCRIPTION

The present disclosure provides a system and a method for affecting, planning and/or designing a space to result in a stimulation of one or more senses of a subject that is located in the space for inducing a desired physiological response to the subject. For example, by applying the output of the system or the method on the space, the space can induce a desired conditional, behavioral, and/or emotional response such as concentration, aggression, calmness, amazement, confusion, etc. One of the unique advantages of the present invention is the ability to provide a variety of solutions for a variety of desired physiological responses due to a designation of each environmental feature to the variety of physiological responses it causes. The physiological response is manifested by a measurable physiological effect. For example, the physiological response can be exhibited by any one of a neuronal response such as the creation of a certain brainwave, hormone secretion, change in heart rate, respiration rate or blood pressure, or any combination thereof. In response to an input of a desired physiological response, environmental features from a pre-obtained data base are selected to be applied in the space to obtain the desired effect. The database includes a list of many environmental features that were examined on subjects and were assigned with the physiological response they induce. Thus, the relevant environmental features from the database that are classified to have the desired effect are selected and an output data indicative of the selected environmental features is outputted to a user or a dedicated system to execute the output data. The output data may include execution profile that may affect on any one of the senses of the subject.

It is to be noted that the environmental features are controllable features in the space that can result in a response of the physiological condition of the subject. The environmental features are categorized by their effect on one of the senses. Namely, environmental feature can be recognized by the subject in the space by sight, scent, touch, hearing or taste.

A first aspect of the present disclosure provides a system for planning, designing, and/or controlling environmental features in a space for enhancing (enhancement of a physiological response can be positive or negative enhancement) or inhibiting at least one physiological response, e.g. conditional, behavioral, and/or emotional response of a subject. The system comprises a processing circuitry, i.e. a controller or control unit, configured for: (i) receiving input data of said desired physiological response, the desired physiological response is selected from a group of a plurality of optional physiological responses, e.g. calmness, concentration, anger, aggression, confusion, etc.; (ii) in response to the received input data, extracting from a pre-obtained database selected environmental features to be applied on the space, the selected environmental features are classified to result in the desired effect. The database contains a long list of environmental features, each environmental features is pre-classified with the effect and its extent it results when applied in the space with respect to a plurality of optional physiological responses; (iii) generating a temporal execution profile of the selected environmental features to thereby obtain the desired physiological response over time; and (iv) outputting an output data indicative of the extracted environmental features.

The processing circuitry is configured to generate said temporal execution profile so as to obtain the desired physiological response in a plurality of time windows along the duration of said temporal execution profile. This can be done based on analysis of the pre-classified environmental features and determination of the processing circuitry how to generate a timeline that includes execution of the selected environmental features in order to obtain the desired physiological response in the subject when he/she are presence in the space where the environmental features are executed.

It is to be noted, that the selected environmental features to be applied to the space, and which are size dependent (e.g. amount of scent particles to be dispensed, audio output profile to be applied in the space, etc.), are referring to a size unit of a space. To apply the environmental features to the size of the actual space, a simple fitting conversion is made, e.g. multiplication of the size of the room with the size of the actual space to obtain the corrected parameters for the application of each environmental feature in the space.

It is to also be noted that any combination of the described embodiments with respect to any aspect of this present disclosure is applicable. In other words, any aspect of the present disclosure can be defined by any combination of the described embodiments.

In some embodiments of the system, the processing circuitry is configured to generate said temporal execution profile based on the effect and the duration that each environmental feature contributes to obtain said desired physiological response. In other words, the processing circuitry is configured to generate one or more time windows, wherein each of the time windows fulfills a certain condition with respect to the desired physiological response.

In some embodiments of the system, each environmental feature in the pre-obtained database is assigned with a scoring rank for enhancing or inhibiting the desired physiological response. The score identifies the extent of the enhancement or inhibition of the physiological response. For example, if an extreme enhancement effect of a physiological response is desired, a relatively high score related environmental feature is to be selected. The processing circuitry is configured to extract environmental features above a selected threshold of said scoring rank according to the desired effect.

In some embodiments of the system, the scoring rank is associated with the enhancement or inhibition of a certain brain wave, i.e. one of delta, theta, alpha, beta, or gamma brain wave. Each environmental feature in the database was tested to determine which brain wave it is stimulated and to what extent. The data on each environmental feature was collected by exposing a subject to the environmental feature and measuring its EEG response. Each response was recorded and according to the known relation between the stimulated respective brain wave and a physiological response, each environmental feature was ranked according to the enhancement it has on the physiological response.

In some embodiments of the system, the environmental features are clustered to sub-categories. The sub-categories can be according to the sense they stimulate or according to the physiological response they enhance or inhibit. The processing circuitry is configured to extract one or more environmental features from each sub-category.

In some embodiments of the system or the method, as described below, each sub-category includes environmental features that are recognizable by or affecting a similar sense. The following are examples of sub-categories of each sense:
(a) Sight sub-category—the sub-category relating to sight includes a plurality of colors and/or a plurality of illumination profiles (types, intensities, wavelengths) that may be applied in the space. In addition, it is to be noted that this category may also include the visibility of the form and/or geometry of objects located within the space. Each environmental feature of the sight sub-category is classified and/or ranked as enhancing or inhibiting a plurality of physiological responses and to what extent.

(b) Hearing sub-category—the sub-category relating to hearing includes a plurality of frequencies, rhythms or tempos and/or intensities of the outputted audio in the space. Each environmental feature of the hearing sub-category is classified and/or ranked as enhancing or inhibiting a plurality of physiological responses and to what extent.

(c) Scent sub-category—the sub-category relating to scent includes a plurality of scent types and/or amount of scent particles to be dispensed in the space. This category may also be obtained by natural plants that are placed in the space to obtain the desired amount of scent. Each environmental feature of the scent sub-category is classified and/or ranked as enhancing or inhibiting a plurality of physiological responses and to what extent.

(d) Touch sub-category—the sub-category relating to touch includes a plurality of temperature ranges, textures, elasticity of objects, transparencies of objects, ornamentals, forms (e.g. round, linear, flat, etc.), dimensions, and/or rigidity of objects to be found in the space. Each environmental feature of the touch sub-category is classified and/or ranked as enhancing or inhibiting a plurality of physiological responses and to what extent.

(e) Taste sub-category—the sub-category relating to taste includes a plurality of food/drink types and their amount to be consumed in the space. Each environmental feature of the taste sub-category is classified and/or ranked as enhancing or inhibiting a plurality of physiological responses and to what extent.

In some embodiments of the system, all environmental features of a sub-category affect one of the senses, selected from: sight, smell, touch, taste, and hearing. Namely, each sub-category includes all the environmental features that affect a certain sense.

In some embodiments of the system, the processing circuitry is configured to generate a temporal execution profile of at least first and second or more environmental features, the first environmental feature is a scent to be dispensed in the space and the second environmental feature is an acoustic output. The execution profile comprises time-depended relation between the first and second environmental features. Namely, the output of the first environmental feature occurs in a certain time relation to the second environmental feature.

In some embodiments of the system, the processing circuitry is configured to generate a temporal execution profile of at least first, second, third or more environmental features. The first environmental feature is a scent to be dispensed in the space, the second environmental feature is an acoustic output and the third is a visual output. The execution profile comprises time-depended relation between the first, second and the third environmental features. Namely, the output of the first environmental feature occurs in a certain time relation to the second and third environmental features.

In some embodiments of the system, the temporal execution profile comprises a periodical scheme that repeats N times in the entire temporal execution profile. Each periodical scheme comprises similar temporal relations between execution of a certain acoustic element and dispensing of scent. It is to be noted that one periodical scheme is not necessarily identical to another periodical scheme but includes similar characteristics. The execution profile is divided into periodical schemes since each physiological response is characterized by a certain effective period and the periodical scheme for each physiological response lasts about the time of said effective period.

In some embodiments of the system, the transition between one periodic scheme to another is performed by an execution of one or more environmental features.

In some embodiments of the system, the temporal execution profile of each periodical scheme comprises acoustic execution profile and scent dispensing profile and a transition execution profile between one periodical scheme to the next one. The transition execution profile can be selected from any one of: acoustic execution profile, execution profile of visual output, scent dispensing execution profile, or any combination thereof. The transition execution profile is different than any execution profile in said N periodical schemes. The difference may be manifested by affecting differently on the physiological state of the subject than the effect on the subject in the periodical schemes. For example, the effect of the transition state on the brain waves activity of the subject can be different than the effect during the periodical scheme.

In some embodiments of the system, the desired physiological response is characterized by a certain score level that is obtained by combination of the selected environmental features from the database. In other words, each environmental features that is selected from the database contributes at a certain amount, according to its pre-assigned scoring, to the score of the desired physiological response and the sum of all the selected environmental features reaches the desired score, which yields the desired physiological response. Thus, the processing circuitry is configured to select the environmental features to obtain the desired score. In some embodiments, the processing circuitry is configured to receive constraints of one or more environmental features that are applied in the space and are not controllable, e.g. light profile or lack of speakers. The processing circuitry is configured to compensate with different types of environmental feature to obtain the desired physiological response, namely the desired score that characterizing said desired physiological response. For example, over expression of touch-related features are applied to a space with no control on the light profile (e.g. constant, non-controllable illumination profile).

In some embodiments of the system, the output data comprises an execution profile of said extracted environmental features. The execution profile my include the output profile of the environmental feature at the space over time.

In some embodiments of the system, the execution profile comprises spatial and/or time depended relation between at least two of the selected environmental features. Namely, the execution profile synchronizes between two execution profiles of two or more environmental features.

In some embodiments of the system, the execution profile comprises a temporal execution profile for each environmental feature. Namely, one environmental feature may be applied at selected times along a timeline, e.g. periodic application of the environmental feature at every selected period time. Each environmental feature may have its own temporal execution profile and the combination of all temporal profiles of the environmental features creates the desired design or scene of the space that enhances or inhibits the desired physiological response. For example, a first environmental feature may be a certain scent that is dispensed from a scent dispenser into the space, a second environmental feature may be an acoustic output of a certain frequency (either a sound, infrasound or an ultrasound frequency) at a certain intensity. The first and the second environmental features are operated according to a time profile and in synchronization with one another. The scent is dispensed in a planned time relation to the acoustic output. This combination yields the desired effect. In some examples, a third environmental feature may be a visual expression in the space. It can either be a constant expression, such as paint of a certain color on the walls or constant illumination profile from a light source; or varying expression such as projected scene of certain shapes and colors in the space. This third environmental feature is applied according to its own temporal execution profile in combination with the first and second environmental features.

In some embodiments of the system, the acoustic output comprises frequencies in the sound range and in the infrasound range.

In some embodiments of the system, the acoustic output is composed of two or more acoustic elements. The acoustic elements are selected from: acoustic frequency, soundscape (namely, the acoustic environment as perceived by humans, in context), music (namely, arranging sounds in time through the elements of melody, harmony, rhythm, and timbre) and sub-conscious signals in ASMR technology. Each of the acoustic elements is associated with enhancement or inhibition of one or more physiological responses. Thus, in order to obtain the desired physiological response, a combination of acoustic elements is to be selected so as to yield said desired physiological response.

In some embodiments of the system, the processing circuitry is configured to select an acoustic output that comprises acoustic frequencies matching the neural oscillations (i.e. brainwaves) associated with the desired physiological response. As noted above, the acoustic frequencies is one acoustic element out of four that are comprised in the acoustic output. While the acoustic frequencies can be categorized with influence on only one brain wave (e.g. alpha, beta, etc.), the other acoustic elements can be categorized with influence on one or more brain waves.

It is to be noted that many physiological responses are classified under each brain wave.

In some embodiments of the system, the processing circuitry is configured to determine a selection of combination of two or more acoustic elements based on the at least one physiological response to be enhanced or inhibited. Each of the acoustic elements is assigned with a score for enhancing or inhibiting a certain physiological response and the processing circuitry is configured to determine a combination of acoustic elements to be outputted together and to generate their execution profile to obtain the desired physiological response.

In some embodiments of the system, at least one of the selected environmental features is a scent to be dispensed in the space. The scent may be originated by dispensing etheric oils, scent extractions, aromatic plants, scent candles, soaps, or any combination thereof.

In some embodiments of the system, the scent to dispensed is selected from synthetic scents and natural scents that are derived by extraction of ethereal oils.

In some embodiments, the system comprising a scent dispenser for dispensing said scent or scent profile.

In some embodiments of the system, the scent dispenser comprises a plurality of scents. The output data comprises dispensing execution data indicative of scent selection and dispensing profile. The scent dispenser is configured to dispense a selected scent at a selected dispensing profile according to the dispensing execution data. In other words, the scent diffuser is in data communication with the processing circuitry and is operated thereby to dispense scents in the space for obtaining the desired physiological response of the subject.

In some embodiments of the system, the processing circuitry is configured to generate a dispensing execution profile that comprises time-depended dispensing profile of synthetic scents and natural scents.

For example, natural scent can be constantly dispensed that causes the subject to maintain over time in a certain state, such as alertness, and a synthetic scent is dispensed periodically to determine a certain rhythm in the space that renders the subject in the space to be more productive.

In some embodiments of the system, at least one of the selected environmental features is an acoustic output.

In some embodiments, the system further comprising an acoustic output unit, e.g. a speaker, a musical instrument, noise isolator or noise cancelling device, acoustic components, or any combination thereof, configured for outputting said acoustic output.

In some embodiments of the system, the output data comprises acoustic execution data indicative of acoustic signal execution profile of said acoustic output. The acoustic output unit is configured for outputting said acoustic execution data to obtain a desired acoustic signal profile. In other words, the acoustic output unit is in data communication with the processing circuitry and is operated thereby, and the is configured to output said acoustic signals profile into the space for obtaining the desired physiological response of the subject. The acoustic signal profile provides a certain tempo, rhythm or beat that enhance or inhibit the physiological response.

In some embodiments of the system, said acoustic signal execution profile is composed of: intensity and/or frequencies profile of the signal.

In some embodiments of the system, at least one of the selected environmental features is a visual output. The visual output can be constant, dynamic, light pattern including specific intensity and colors, etc. For example, the visual input can be the selection of a certain illumination to be illuminated by a light source in the space. The illumination is characterized by a certain wavelength range and a certain intensity range. The illumination profile may vary over time.

In some embodiments of the system, the visual output is constant in time. In some embodiments, the visual output is varying over time. It is to be noted that the visual output can a combination of constant and varying illumination profiles.

In some embodiments, the system further comprising a visual output unit, e.g. a projector, light sources, a display, or any combination thereof, for outputting said visual output.

In some embodiments of the system, the output data comprises visual execution data indicative of the desired visual profile to be visual. The profile refers to the intensity of the visual output, its pattern, the temporal sequence of its execution, etc. The visual output unit is configured to execute said visual execution data to obtain the desired visual profile.

In some embodiments of the system, at least one of the selected environmental features is a touch-related feature.

In some embodiments of the system, the touch-related feature is selected from: texture of different objects in the space, temperature in the space, humidity conditions, elasticity of objects in the space, transparency degree of objects in the space, shape and size of objects in the space, weight of objects in the space, or any combination thereof. It is to be noted that objects in the space also refer to object defining the space such as walls, floor, ceiling, etc.

In some embodiments of the system, at least one of the selected environmental features is a taste-related feature.

In some embodiments of the system, said taste-related feature comprises instructions for types of foods and drinks to be consumed in the space and types foods and drinks that to be prevented from consumption in that space. The selection of certain food and/or drinks is based on their tested effect for resulting in the desired physiological response.

In some embodiment of the system, the space is any one of a bedroom, workspace, classroom, conference room, playground, therapeutic-related space, kindergarten, library, sales room/office, management office, hospitals, clinics, sport spaces, gym, home-living spaces, dining rooms, vehicles (such as cars, airplanes, trains, bars), coffee houses, restaurants, cultural-related spaces, gas stations, natural reserves, religion-related spaces, parks, parking lots, roads, smoking areas, virtual spaces (such as applications, websites), etc.

In some embodiments of the system, the physiological response comprises wakefulness, alertness, concentration, awareness, amazement, confusion, love, patriotism, sympathy, courage, optimism, happiness, authenticity, unity, responsibility, balance, intuition, intimacy, caring, trust, faith, artistic thinking, empathy, humanity, assertiveness, aesthetics, nobility, clarity, decisiveness, security, self-expression, pride, heroism, diversity, flexibility, accuracy, distinction, understanding, logic, fairness, protection, fulfillment, mutuality, delivery, humor, thanksgiving, identification, achievements, peacefulness, joy, ecstasy, reception, love, trust, admiration, surrender, optimism, interest, expectation, distraction, wonder, respect, fear, terror, discomfort, reflection, sadness, grief, remorse, boredom, disgust, loathing, contempt, anger, wrath, aggressiveness, vigilance, excitement, high spirit, satisfaction, calmness, relaxation, depression, confusion, pressure, nervousness, tension, etc.

In some embodiments of the system, the physiological response comprises causing a change in the subject in at least one of: certain brain wave activity, hormone secretion, such as adrenaline, noradrenaline, oxytocin, dopamine, melatonin, serotonin, etc., heart rate, body temperature, blood pressure, pupil dilation, respiratory rate or any combination thereof.

Another aspect of the present disclosure provides a method for planning and/or controlling environmental features in a space for enhancing or inhibiting at least one physiological response, e.g. conditional, behavioral, and/or emotional response, of a subject. The method includes (i) receiving input data of said desired physiological response, e.g. calmness, concentration, anger, aggression, confusion, etc.; (ii) in response to the input data, selecting environmental features, e.g. from a pre-obtained database, to be applied on the space. The selected environmental features are classified to result in the desired physiological response when applied in a space; (iii) generating a temporal execution profile of the selected environmental features so as to obtain the desired physiological response over time; and (iv) outputting an output data indicative of the selected environmental features. The output data includes instructions for applying the selected environmental features in the space or execution data to be executed by a dedicated system to apply the environmental features in the space and obtain the desired physiological response for a subject being present in the space.

In some embodiments, the method comprises scoring each environmental feature available for selection for enhancing or inhibiting each available physiological response for selection. This can be performed by measuring physiological parameters of one or more test subjects in response to an exposure for the environmental feature. For example, the physiological parameter can be brain activity response, e.g. enhancement or inhibition of a certain brain wave, change is heart rate, blood pressure, hormone secretion, etc. In some embodiments of the method, the environmental features are selected from a pre-obtained database.

In some embodiments, the method comprising assigning each environmental feature with a scoring rank for enhancing or inhibiting the desired physiological response. Said selecting step of the method comprises selecting environmental features above a selected threshold of said scoring rank according to the desired physiological response.

In some embodiments, the method comprising exposing one or more test subjects to each optional environmental feature and recording the profile of physiological parameters of said one or more subjects before and following the exposure to the environmental feature. The method further comprising assigning a scoring rank for enhancing or inhibiting the desired physiological response based on the recorded physiological parameters. The physiological parameters can be selected from: effect on brain waves activity of the subject, heart rate, blood pressure, hormone secretion, respiration rate, etc. The physiological parameters of the subject are being indicative of the physiological response of the subject to the exposure of the environmental feature.

In some embodiments of the method, said scoring rank is associated with the enhancement or inhibition of a certain brain wave, i.e. one of delta, theta, alpha, beta, or gamma brain wave. Each environmental feature in the database was tested to determine which brain wave it is stimulated and to what extent. The data on each environmental feature was collected by exposing a subject to the environmental feature and measuring its EEG response. Each response was recorded and according to the known relation between the stimulated respective brain wave and a physiological response, each environmental feature was ranked according to the enhancement it has on the physiological response.

In some embodiments of the method, the environmental features are clustered to sub-categories. The method further comprising selecting one or more environmental features from each sub-category, wherein each sub-category consists environmental features that are recognizable by or affecting a similar sense.

In some embodiments of the method, all environmental features of a sub-category affect one of the senses, selected from: sight, smell, touch, taste, and hearing.

In some embodiments of the method, the desired physiological response is characterized by a certain score level that is obtained by a combination of the selected environmental features from the database. In other words, each environmental features that is selected from the database contributes at a certain amount, according to its pre-assigned scoring, to the score of the desired physiological response and the sum of all the selected environmental features reaches the desired score, which yields the desired physiological response. Thus, the method includes selecting the environmental features to obtain the desired score. In some embodiments, the method further includes (i) receiving constraints of one or more environmental features that are applied in the space and are not controllable, e.g. light profile or lack of speakers; and (ii) selecting other controllable environmental feature to compensate the score of the non-controllable environmental feature to obtain the desired physiological response, namely the desired score that characterizing said desired physiological response. For example, over expression of touch-related features are applied to a space with no control on the light profile (e.g. constant and non-controllable illumination profile).

In some embodiments of the method, said output data comprises an execution profile of said selected environmental features.

In some embodiments, the method comprising generating a temporal execution profile of at least first and second or more environmental features, the first environmental feature is a scent to be dispensed in the space and the second environmental feature is an acoustic output. The execution profile comprises time-depended relation between the first and second environmental features. Namely, the output of the first environmental feature occurs in a certain time relation to the second environmental feature.

In some embodiments, the method comprising generating a temporal execution profile of at least first, second, third or more environmental features. The first environmental feature is a scent to be dispensed in the space, the second environmental feature is an acoustic output and the third is a visual output. The execution profile comprises time-depended relation between the first, second and the third environmental features. Namely, the output of the first environmental feature occurs in a certain time relation to the second and third environmental features.

In some embodiments of the method, the temporal execution profile comprises a periodical scheme that repeats N times in the entire temporal execution profile. Each periodical scheme comprises similar temporal relations between execution of a certain acoustic element and dispensing of scent. It is to be noted that one periodical scheme is not necessarily identical to another periodical scheme but includes similar characteristics. The execution profile is divided into periodical schemes since each physiological response is characterized by a certain effective period and the periodical scheme for each physiological response lasts about the time of said effective period.

In some embodiments of the method, the transition between one periodic scheme to another is performed by an execution of one or more environmental features.

In some embodiments of the method, the temporal execution profile of each periodical scheme comprises acoustic execution profile and scent dispensing profile and a transition execution profile between one periodical scheme to the next one. The transition execution profile can be selected from any one of: acoustic execution profile, execution profile of visual output, scent dispensing execution profile, or any combination thereof. The transition execution profile is different than any execution profile in said N periodical schemes. The difference may be manifested by affecting differently on the physiological state of the subject than the effect on the subject in the periodical schemes. For example, the effect of the transition state on the brain waves activity of the subject can be different than the effect during the periodical scheme.

In some embodiments of the method, said execution profile comprises spatial and/or time depended relation between at least two of the selected environmental features.

In some embodiments of the method, said execution profile comprises a temporal execution profile for each environmental feature. Namely, one environmental feature may be applied at selected times along a timeline, e.g. periodic application of the environmental feature every selected period time. Each environmental feature may have its own temporal execution profile and the combination of all temporal profiles of the environmental features creates the desired design or scene of the space that enhances or inhibits the desired physiological response. For example, a first environmental feature may be a certain scent that is dispensed from a scent dispenser into the space, a second environmental feature may be an acoustic output of a certain frequency (either a sound, infrasound or an ultrasound frequency) at a certain intensity. The first and the second environmental features are operated according to a time profile and in synchronization with one another. The scent is dispensed in a planned time relation to the acoustic output. This combination yields the desired effect. In some examples, a third environmental feature may be a visual expression in the space. It can either be a constant expression, such as paint of a certain color on the walls or varying expression such as projected scene of certain shapes and colors in the space. This third environmental feature is applied according to its own temporal execution profile in combination with the first and second environmental features.

In some embodiments of the method, at least one of the selected environmental features is a scent to be dispensed in the space. In some embodiments, the method further comprising dispensing said scent.

In some embodiments of the method, said output data comprises dispensing execution data indicative of scent selection and dispensing profile. The method further comprising dispensing scent into the space based on the dispensing profile.

In some embodiments of the method, at least one of the selected environmental features is an acoustic output. In some embodiments, the method further comprising outputting said acoustic output.

In some embodiments of the method, said output data comprises acoustic execution data indicative of acoustic signal execution profile of said acoustic output. The method further comprising outputting said acoustic execution data to obtain a desired acoustic signal profile. The acoustic signal profile provides a certain tempo, rhythm or beat that enhance or inhibit the physiological response.

In some embodiments, the method comprising generating a dispensing execution profile that comprises time-depended dispensing profile of synthetic scents and natural scents.

For example, natural scent can be constantly dispensed that causes the subject to maintain over time in a certain state, such as alertness, and a synthetic scent is dispensed periodically to determine a certain rhythm in the space that renders the subject in the space to be more productive

In some embodiments of the method, said acoustic signal profile is composed of: intensity and/or frequencies profile of the signal.

In some embodiments of the method, at least one of the selected environmental features is a visual output. The visual output can be constant illumination pattern, dynamic illumination pattern, a selection of range of illumination wavelengths and a selected intensity.

In some embodiments of the method, the visual output is constant in time.

In some embodiments the visual output is varying over time. It is to be noted that the visual output can a combination of constant and varying illumination profiles.

In some embodiments, the method further comprising outputting said visual output.

In some embodiments of the method, said output data comprises visual execution data indicative of the desired visual profile to be visual. The profile refers to the intensity of the visual output, its pattern, the temporal sequence of its execution, etc. The method further comprising outputting said desired visual profile.

In some embodiments of the method, at least one of the selected environmental features is touch-related feature.

In some embodiments of the method, the touch-related feature is selected from: texture of different objects in the space, temperature in the space, humidity conditions, elasticity of objects in the space, transparency degree of objects in the space, shape and size of objects in the space, weight of objects in the space, or any combination thereof. It is to be noted that objects in the space also refer to object defining the space such as walls, floor, ceiling, etc.

In some embodiments of the method, at least one of the selected environmental features is taste-related feature.

In some embodiments of the method, wherein said taste-related feature comprises instructions for types of foods and drinks to be consumed in the space and types foods and drinks that to be prevented from consumption in that space.

In some embodiments of the method, the space is any one of a bedroom, workspace, classroom, conference room, playground, therapeutic-related space, kindergarten, library, sales room/office, management office, hospitals, clinics, sport spaces, gym, home-living spaces, dining rooms, vehicles (such as cars, airplanes, trains, bars), coffee houses, restaurants, cultural-related spaces, gas stations, natural reserves, religion-related spaces, parks, parking lots, roads, smoking areas, virtual spaces (such as applications, websites), etc.

In some embodiments of the method, the physiological response comprises wakefulness, alertness, concentration, amazement, aggression, awareness, etc.

In some embodiments of the method, the physiological response comprises causing a change in the subject in at least one of: certain brain wave activity, hormone secretion such as adrenaline, noradrenaline, oxytocin, dopamine, melatonin, serotonin, etc., heart rate, body temperature, blood pressure, pupil dilation, respiratory rate or any combination thereof.

### Embodiments

The following are optional embodiments and combinations thereof in accordance with aspects of the present disclosure:
1. A system for affecting, planning and/or controlling environmental features in a space for enhancing or inhibiting at least one physiological response of a subject, comprising:
   a processing circuitry configured for
      (i) receiving input data of said desired physiological response out of a plurality of optional physiological responses;
      (ii) in response to the received input data, extracting from a pre-obtained database selected environmental features to be applied on the space, said selected environmental features are classified to result in the desired effect, and generating temporal execution profile of said selected environmental features;
      (iii) outputting an output data indicative of the extracted environmental features that comprises said execution profile.
2. The system of embodiment 1, wherein each environmental feature in the pre-obtained database is assigned with a scoring rank for enhancing or inhibiting the desired physiological response, the processing circuitry is configured to extract environmental features above a selected threshold of said scoring rank.
3. The system of embodiment 2, wherein said scoring rank is associated with the enhancement or inhibition of a certain brain wave.
4. The system of any one of embodiments 1-3, wherein the environmental features are clustered to sub-categories, the processing circuitry is configured to extract one or more environmental features from each sub-category.
5. The system of embodiment 4, wherein each sub-category consists environmental features that are recognizable by or affecting a similar sense.
6. The system of embodiment 4 or 5, wherein all environmental features of a sub-category affect one of the senses, selected from: sight, smell, touch, taste, and hearing.
7. The system of any one of embodiments 1-6, wherein said execution profile comprises spatial and/or time dependent-relation between at least two of the selected environmental features.
8. The system of embodiment 7, wherein said execution profile comprises a time-dependent execution profile for two or more selected environmental features.
9. The system of embodiment 8, wherein at least one of the selected environmental features is a scent to be dispensed in the space and at least one of the selected environmental features is an acoustic output.
10. The system of embodiment 9, wherein said execution profile comprises N periodical schemes, the execution profile in one periodical scheme comprises similar time-depended relations of executions of said at least two selected environmental features.
11. The system of embodiment 10, wherein each two following periodical schemes are separated by a transition scheme that comprises a transition execution profile of at least one environmental feature different than the execution profile in said N periodical schemes.
12. The system of any one of embodiments 1-11, wherein at least one of the selected environmental features is a scent to be dispensed in the space, the scent is selected from synthetic scent, natural scent, or a combination thereof.
13. The system of embodiment 12, comprising a scent dispenser for dispensing said scent.
14. The system of embodiment 13, wherein said scent dispenser comprises a plurality of scents, and said temporal execution profile comprises dispensing execution data indicative of scent selection and dispensing profile, said scent dispenser is configured to dispense a selected scent at a selected dispensing profile according to the dispensing execution data.
15. The system of any one of embodiments 1-14, wherein at least one of the selected environmental features is an acoustic output.
16. The system of embodiment 15, comprising an acoustic output unit configured for outputting said acoustic output.
17. The system of embodiment 16, wherein said temporal execution profile comprises acoustic execution data indicative of acoustic signal execution profile of said acoustic output and said acoustic output unit is configured for outputting said acoustic execution data to obtain a desired acoustic signal profile.
18. The system of embodiment 17, wherein said acoustic signal profile is composed of: intensity and/or frequencies profile of the signal.
19. The system of any one of embodiments 15-18, wherein said acoustic output is composed of two or more acoustic elements selected from: acoustic frequency, soundscape, music, and sub-conscious signals in ASMR technology, wherein each of the two or more acoustic elements is associated with effect on said physiological response.
20. The system of embodiment 19, wherein the processing circuitry is configured to combine two or more acoustic elements to generate said acoustic output to obtain said physiological response.
21. The system of any one of embodiments 15-20, wherein the processing circuitry is configured to select an acoustic output that comprises acoustic frequencies matching the neural oscillations associated with the desired physiological response.
22. The system of any one of embodiments 1-21, wherein at least one of the selected environmental features is a visual output.
23. The system of embodiment 22, wherein the visual output is constant in time.
24. The system of embodiment 22, wherein the visual output is varying over time.
25. The system of any one of embodiments 22-24, comprising a visual output unit for outputting said visual output.
26. The system of embodiment 25, wherein said output data comprises visual execution data indicative of the desired visual profile to be visual, said visual output unit is configured to execute said visual execution data to obtain the desired visual profile.
27. The system of any one of embodiments 1-26, wherein at least one of the selected environmental features is a touch-related feature.
28. The system of embodiment 27, wherein said touch-related feature is selected from: texture of different objects in the space, temperature in the space, humidity conditions, elasticity of objects in the space, transparency degree of objects in the space, shape and size of objects in the space, weight of objects in the space, or any combination thereof
29. The system of any one of embodiments 1-28, wherein at least one of the selected environmental features is a taste-related feature.
30. The system of embodiment 29, wherein said taste-related feature comprises instructions for types of foods and drinks to be consumed in the space and types of foods and drinks that to be prevented from consumption in that space.
31. The system of any one of embodiments 1-30, wherein the space is selected from any one of the group consisting of: a bedroom, workspace, classroom, conference room, playground, therapeutic-related space, kindergarten, library, sales room/office, management office, hospitals, clinics, sport spaces, gym, home-living spaces, dining rooms, vehicles, coffee houses, restaurants, cultural-related spaces, gas stations, natural reserves, religion-related spaces, parks, parking lots, roads, smoking areas, virtual spaces.
32. The system of any one of embodiments 1-31, wherein the physiological response comprises wakefulness, alertness, amazement, concentration, awareness, confusion.
33. The system of any one of embodiments 1-32, wherein the physiological response comprises causing a change in the subject in at least one of: certain brain wave activity, hormone secretion, heart rate, body temperature, blood pressure, pupil dilation, respiratory rate or any combination thereof
34. A method for affecting, planning and/or controlling environmental features in a space for enhancing or inhibiting at least one physiological response of a subject, comprising:
   receiving input data of said desired physiological response;
   based on the input data, selecting environmental features to be applied on the space, said selected environmental are classified to result in the desired physiological response;
   generating a temporal execution profile of said selected environmental features to obtain said physiological response;
   outputting an output data indicative of the selected environmental features and the temporal execution profile.
35. The method of embodiment 34, wherein said environmental features are selected from a pre-obtained database.
36. The method of embodiment 34 or 35, comprising assigning each environmental feature with a scoring rank for enhancing and/or inhibiting the desired physiological response, said selecting comprises selecting environmental features above a selected threshold of said scoring rank.
37. The method of embodiment 36, wherein said scoring rank is associated with the enhancement or inhibition of a certain brain wave.
38. The method of any one of embodiments 34-37, wherein the environmental features are clustered to sub-categories, the method comprising selecting one or more environmental features from each sub-category, wherein each sub-category consists environmental features that are recognizable by or affecting a similar sense.
39. The method of embodiment 38, wherein all environmental features of a sub-category affect one of the senses, selected from: sight, smell, touch, taste, and hearing.
40. The method of embodiment 39, wherein said execution profile comprises spatial and/or time-dependent relation between at least two of the selected environmental features.
41. The method of embodiment 39 or 40, wherein said execution profile comprises a time-dependent execution profile of two or more selected environmental features.
42. The method of embodiment 40 or 41, wherein at least one of the selected environmental features is a scent to be dispensed in the space and at least one of the selected environmental features is an acoustic output.
43. The method of embodiment 42, wherein said execution profile comprises N periodical schemes, the execution profile in one periodical scheme comprises similar time-depended relations of executions of said at least two selected environmental features.
44. The method of embodiment 43, wherein each two following periodical schemes are separated by a transition scheme that comprises a transition execution profile of at least one environmental feature different than the execution profile in said N periodical schemes.
45. The method of any one of embodiments 34-44, wherein at least one of the selected environmental features is a scent to be dispensed in the space, the scent is selected from synthetic scent, natural scent, or a combination thereof.
46. The method of embodiment 45, comprising dispensing said scent.
47. The method of embodiment 46, wherein said temporal execution profile comprises dispensing execution data indicative of scent selection and dispensing profile, the method further comprising dispensing scent into the space based on the dispensing profile.
48. The method of any one of embodiments 34-47, wherein at least one of the selected environmental features is an acoustic output.
49. The method of embodiment 48, comprising outputting said acoustic output.
50. The method of embodiment 49, wherein said temporal execution profile comprises acoustic execution data indicative of acoustic signal execution profile of said acoustic output, the method further comprising outputting said acoustic execution data to obtain a desired acoustic signal profile.
51. The method of embodiment 50, wherein said acoustic signal profile is composed of: intensity and/or frequencies profile of the signal.
52. The method of any one of embodiments 48-51, wherein said acoustic output is composed of two or more acoustic elements selected from: acoustic frequency, soundscape, music, and sub-conscious signals in ASMR technology, wherein each of the two or more acoustic elements is associated with effect on said physiological response, the method further comprising.
53. The method of embodiment 52, combining two or more acoustic elements to generate said acoustic output to obtain said physiological response.
54. The method of any one of embodiments 48-53, comprising generating an acoustic output that comprises acoustic frequencies matching the neural oscillations associated with the desired physiological response.
55. The method of any one of embodiments 34-54, wherein at least one of the selected environmental features is a visual output.
56. The method of embodiment 55, wherein the visual output is constant in time.
57. The method of embodiment 55, wherein the visual output is varying over time.
58. The method of any one of embodiments 55-57, comprising outputting said visual output.
59. The method of embodiment 58, wherein said output data comprises visual execution data indicative of the desired visual profile to be visual, the method further comprising outputting said desired visual profile.
60. The method of any one of embodiments 34-59, wherein at least one of the selected environmental features is touch-related feature.
61. The method of embodiment 60, wherein said touch-related feature is selected from: texture of different objects in the space, temperature in the space, humidity conditions, elasticity of objects in the space, transparency degree of objects in the space, shape and size of objects in the space, weight of objects in the space, or any combination thereof
62. The method of any one of embodiments 34-61, wherein at least one of the selected environmental features is taste-related feature.
63. The method of embodiment 62, wherein said taste-related feature comprises instructions for types of foods and drinks to be consumed in the space and types of foods and drinks that to be prevented from consumption in that space.
64. The method of any one of embodiments 34-63, wherein the space is any one of bedroom, a car, a common workspace, a classroom.
65. The method of any one of embodiments 34-64, wherein the physiological response comprises wakefulness, alertness, amazement, concentration, awareness, confusion.
66. The method of any one of embodiments 34-65, wherein the physiological response comprises causing a change in the subject in at least one of: certain brain wave activity, hormone secretion, heart rate, body temperature, blood pressure, pupil dilation, respiratory rate or any combination thereof.
67. The method of any one of embodiments 34-65, comprising (i) exposing one or more test subjects to each optional environmental feature and recording physiological parameters of said one or more subjects following the exposure to the environmental feature, and (ii) assigning a scoring rank for enhancing or inhibiting the desired physiological response based on the recorded physiological parameters.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are block diagrams of non-limiting examples of different embodiments of the system of the present disclosure.

FIG. 2 is a transverse cross section of an exemplary illustration of a space to be planned for inducing a desired physiological response of a subject present therein.

FIG. 3 is a flow diagram exemplifying a non-limiting embodiment of the method according to an aspect of the present disclosure.

### DETAILED DESCRIPTION OF EMBODIMENTS

The following figures are provided to exemplify embodiments and realization of the invention of the present disclosure.

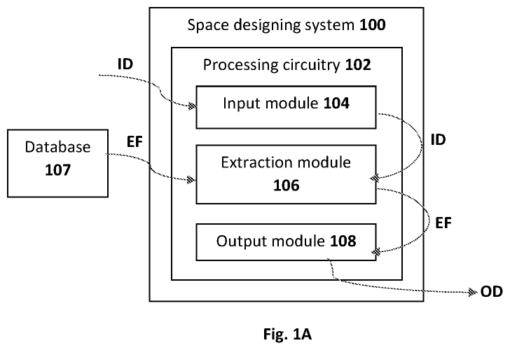

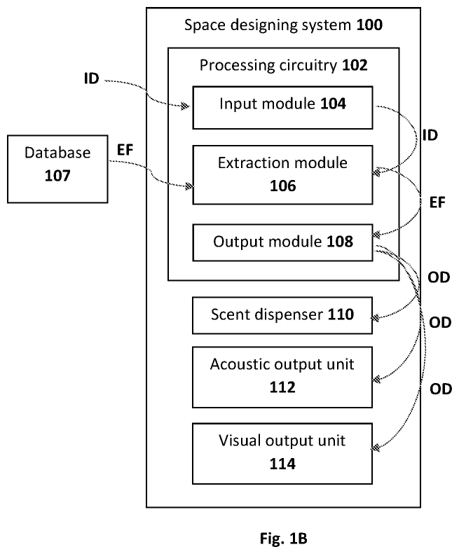

Reference is made to FIGS. 1A-1B, which are block diagrams exemplifying different embodiments of the space designing system for planning and/or controlling environmental features in a space for enhancing or inhibiting at least one physiological response, e.g. conditional, behavioral, and/or emotional response, of a subject that enters or present in the space. FIG. 1A exemplifies an embodiment of the space designing system 100 that includes a processing circuitry 102, i.e. a processor that can be centralized or distributed. The processing circuitry 102 comprises an input module 104 for receiving input data ID indicative of the desired physiological response to be obtained in the space. The input data ID is received in the system by an input of a user, e.g. by a dedicated user interface for the system.

It is to be noted that a space may be an indoor space, outdoor space, or a virtual space. The following are examples of spaces to be affected by the system or the method of the present disclosure: a classroom, conference room, playground, therapeutic-related space, kindergarten, library, sales room/office, management office, hospitals, clinics, sport spaces, gym, home-living spaces, dining rooms, vehicles (such as cars, airplanes, trains, bars), coffee houses, restaurants, cultural-related spaces, gas stations, natural reserves, religion-related spaces, parks, parking lots, roads, smoking areas, virtual spaces (such as applications, websites), etc.

There is no requirement for the input data to include any data relating the space to be affected by the system. The system is configured to output generic solutions relevant for any space and the user may select the relevant environmental features to be applied on the space out of a variety of environmental features that are outputted by the system as detailed below.

The processing circuitry further includes an extraction module 106 that is configured to receive the input data ID and based thereon extract from a pre-obtained database 107 relevant environmental features EF that are classified as suitable to yield the desired physiological response on the subject when they are applied in a space that the subject is present in.

The pre-obtained database 107 includes a plurality of environmental features, each is classified as an enhancer or inhibitor of at least one physiological response. An environmental feature is any feature that affect on at least one of the senses of humans, e.g. scent, visual pattern, sound, texture, or taste. Each environmental feature may be classified to affect more than one physiological response and for each physiological response the extent of the influence of any environmental feature is assigned thereto. For example, if you an extreme physiological response is required, a first environmental feature can be applied and for a moderate physiological response, a second environmental feature is applied. The collection of the data pieces in the database for the classification of the environmental features was performed by measuring physiological responses of subjects while being exposed to the environmental feature. The physiological responses may be any one of: respiration rate, heart rate, hear rate variability, blood pressure, pupil size, gaze direction profile and/or facial expressions by image analysis of the face of the subject, galvanic skin response (GSR), engagement profile of the user (namely the activity profile of the subject in the space), neural oscillations such as EEG measurements of brains waves, hormone secretion, etc. The environmental features were isolated and examined independently to realize the specific effect of each environmental feature. For example, the effect of a sound in a certain frequency was tested to realize the brains wave it induces. The effect of the sound was tested with respect to different intensities and different repetition rates. A similar test was performed for any of the environmental features that are stored in the database.

The processing circuitry 102 generates a temporal execution profile of extracted environmental features EF that is transmitted to an output module 108. The output module 108 is configured to output an output data OD indicative of or comprises the temporal execution profile of the extracted environmental features to be applied in the space. The output data OD may be (i) in a passive form, i.e. planning instructions to the user which environmental features to apply in the space and their profile of application; and/or (ii) in an active form, i.e. execution commands for components that are present in the space and are configured to provide senses-stimulating output into the space, e.g. scents, sounds, light patterns, etc. The execution commands operate the components in the desired manner to obtain the desired physiological response of the subject in the space.

FIG. 1B differs from FIG. 1A by that the system includes several components for executing the output data to affect the space for obtaining the desired physiological response. In this embodiment, a scent dispenser 110, an acoustic output unit 112, e.g. a loudspeaker, and a visual output unit 114, e.g. a projector or illumination means such light sources, are included in the system 100. It is to be noted that any combination of one or more of the scent dispenser, acoustic output unit and visual output unit can be used in the system and it is to be understood that the system does not require the use of the three together in every scenario. For the ease of explanation, the three components are described together in the system.

The output module 108 is configured to transmit the output data OD to the scent dispenser 110, acoustic output unit 112 and visual output unit, the output data OD comprises data pieces that are relevant for each of the components, namely execution commands to be executed by the component. The execution commands may include a temporal profile of the desired effect by the component, for example to dispense a certain scent at a desired frequency, project a certain illumination pattern on a wall or in a display at desired time windows, applying certain illumination type in the space, e.g. illumination wavelength range and/or intensity of illumination, output an acoustic signal (in the sound, ultrasound or infrasound range) at a certain intensity and at certain time windows, etc.

In some embodiments, the system is configured for planning and/or controlling the design of two adjacent spaces, each space is intended to induce different physiological response on a subject present therein. The relation between the spaces may increase the physiological response of a subject. For example, if the subject moves from one space to the other, a contrast between the environmental conditions in the two spaces may increase the physiological response of the subject.

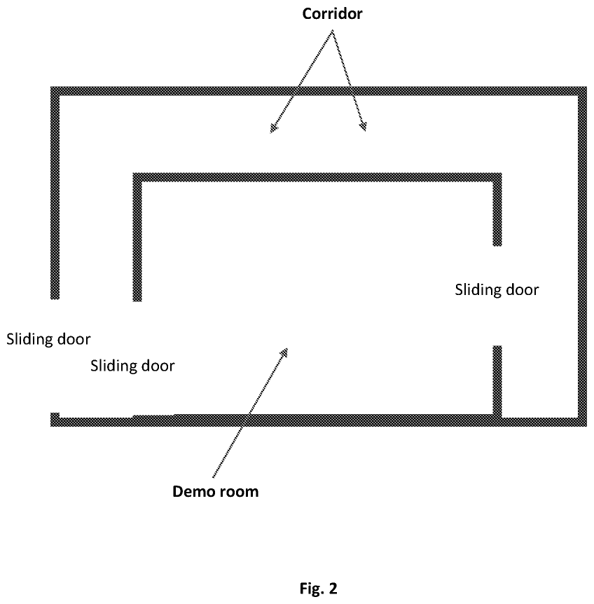

FIG. 2 shows a transverse cross section of an illustration of a space to be planned to induce desired physiological responses of subject being present in the space. The space is intended to be used for displaying in a convention and is divided into two sub-spaces: a pre-show corridor and a demo room. The space is required to induce amazement, which is obtained by inducing surprising and curiosity effect on a subject. The system is inputted with the requirements for the space and generate an output data indicative of environmental features to be applied on the space to obtain the desired physiological response. The general appearance of each sub-space is a contrast of the other—the corridor is dark, narrow, and low and the demo room is very bright and bigger than the corridor with effects that give a feeling of a much bigger room. The contrast between the sub-spaces creates an experience that results in secretion of dopamine, which makes the subject to feel in euphoria and excitement. Each sub-space is planned with environmental features that are selected to enhance the desired physiological response therein by stimulating each of the senses at a very specific way, as detailed below:

### Hearing

Corridor—a sound characterized by a frequency of 18 Hz is selected in a rhythm of 60-125 bpm. The intensity of the sound is selected to be between 60 dB to 100 dB.

Demo room—a sound characterized by a frequency of 40 Hz in a rhythm of 10 bpm. The intensity of the sound is selected to be about 10 dB.

### Sight

Corridor—illumination of light of color of 1500K, intensity of 50 LUX with an illumination angle of 12° above the horizontal plane.

The colors of the corridor are selected to be dark grey/graphite with lit color spots and displays displaying exhibition of clocks, physical and digitals to create tension in the users.

The geometry is selected to form a straight and low sub-space that motivate to move in the forward direction.

Demo room—illumination of 360° of light of color of 4500K, intensity of 350 LUX.

The colors in this room are white and some portions of the walls are covered with mirrors. This combination induces a feeling of levitation and disorientation that results in a feeling of amazement. The geometry of the demo room is selected to be rounded.

### Touch

Corridor—the temperature is selected to be 18° C. The walls are selected to be of a rough texture and the floor is made of concrete.

Demo room—the temperature is selected to be 22° C. The texture of the walls is selected to have a smooth texture. The floor is made of a pliable polymer material to grant a soft feeling while stepping on the floor.

### Scent

Corridor—oily extraction of eucalyptus is sprayed by a plurality of scent dispensers in a frequency of 1 spraying every minute.

Demo room—continuous dispensing of extraction of combination of lavender, Melissa and cotton through the air conditioning system into the sub-space.

### Taste

Demo room—combination of sweet and spicy drinks of 240 ml.

The selected environmental features that are sensible by at least one of the senses are selected from the database based on the score rank of each feature for enhancing a physiological response of amazement or amazement-related physiological response.

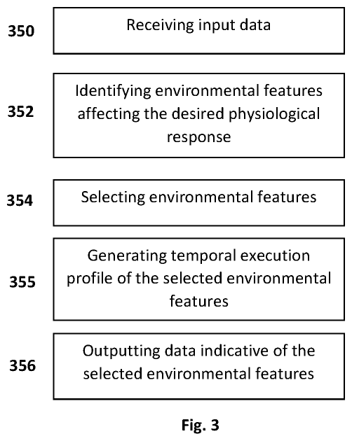

Reference is now made to FIG. 3, which is a flow diagram of the method for planning and/or controlling environmental features in a space for enhancing or inhibiting at least one physiological response, e.g. conditional, behavioral, and/or emotional response, of a subject. The method includes receiving input data 350 that comprises information of the physiological response in a subject to be obtained when being present in the space. The input data can include the extent of the physiological response or a combination of several physiological responses together. Optionally, the input data may include information of the space, e.g. its geometrical dimensions, type/purpose, geographical location, etc. The method further includes identifying, in a pre-obtained database, suitable environmental features affecting the desired physiological response 352. The environmental features being identified are that pre-classified to have the desired effect and/or assigned with a score rank above a certain threshold with respect to the desired physiological response. Upon identifying the environmental features, the method further includes selecting said suitable environmental features 354 and generating a temporal execution profile of said selected environmental features 355. Then, the method further comprising outputting data indicative of or comprises the temporal execution profile of the selected environmental features 356 to be used either by a user or a dedicated system that is configured to execute the output data. The output data may include information for applying the environmental features in the space. For example, the output data may include dispensing a certain scent profile in the space, applying a certain illumination profile in the space, outputting a certain audio/acoustic profile, etc. Once the selected environmental features are applied in the space, a subject that present in the space experiencing enhancement or inhibition of a physiological parameter according to the desired physiological response of the input data.

1. A system for affecting, planning and/or controlling environmental features in a space for enhancing or inhibiting at least one physiological response of a subject, comprising:
a processing circuitry configured for
(i) receiving input data of said desired physiological response out of a plurality of optional physiological responses;
(ii) in response to the received input data, extracting from a pre-obtained database selected environmental features to be applied on the space, said selected environmental features are classified to result in the desired effect, and generating temporal execution profile of said selected environmental features;
(iii) outputting an output data indicative of the extracted environmental features that comprises said execution profile.

2. The system of claim 1, wherein each environmental feature in the pre-obtained database is assigned with a scoring rank for enhancing or inhibiting the desired physiological response, the processing circuitry is configured to extract environmental features above a selected threshold of said scoring rank;
wherein said scoring rank is associated with the enhancement or inhibition of a certain brain wave;
wherein the environmental features are clustered to sub-categories, each sub-category consists environmental features that are recognizable by or affecting a similar sense, the processing circuitry is configured to extract one or more environmental features from each sub-category;
wherein all environmental features of a sub-category affect one of the senses, selected from: sight, smell, touch, taste, and hearing.

3.-5. (canceled)

6. The system of claim 1, wherein said execution profile comprises spatial and/or time dependent-relation between at least two of the selected environmental features;
wherein said execution profile comprises a time-dependent execution profile for two or more selected environmental features;
wherein at least one of the selected environmental features is a scent to be dispensed in the space and at least one of the selected environmental features is an acoustic output, wherein the scent is selected from synthetic scent, natural scent, or a combination thereof.

7.-8. (canceled)

9. The system of claim 2, wherein said execution profile comprises N periodical schemes, the execution profile in one periodical scheme comprises similar time-depended relations of executions of said at least two selected environmental features.

10. The system of claim 2, wherein each two following periodical schemes are separated by a transition scheme that comprises a transition execution profile of at least one environmental feature different than the execution profile in said N periodical schemes.

11. (canceled)

12. The system of claim 9, comprising a scent dispenser for dispensing said scent;
wherein said scent dispenser comprises a plurality of scents, and said temporal execution profile comprises dispensing execution data indicative of scent selection and dispensing profile, said scent dispenser is configured to dispense a selected scent at a selected dispensing profile according to the dispensing execution data.

13. (canceled)

14. The system of claim 1, comprising an acoustic output unit;
wherein at least one of the selected environmental features is an acoustic output; and
wherein said temporal execution profile comprises acoustic execution data indicative of acoustic signal execution profile of said acoustic output and said acoustic output unit is configured for outputting said acoustic execution data to obtain a desired acoustic signal profile;
wherein said acoustic signal profile is composed of intensity and/or frequencies profile of the signal.

15.-17. (canceled)

18. The system of claim 14, wherein said acoustic output is composed of two or more acoustic elements selected from: acoustic frequency, soundscape, music, and sub-conscious signals in autonomous sensory meridian response (ASMR) technology, wherein each of the two or more acoustic elements is associated with effect on said physiological response.

19. The system of claim 14, wherein the processing circuitry is configured to combine two or more acoustic elements to generate said acoustic output to obtain said physiological response.

20. The system of claim 14, wherein the processing circuitry is configured to select an acoustic output that comprises acoustic frequencies matching the neural oscillations associated with the desired physiological response.

21. The system of claim 1, comprising a visual output unit, wherein said output data comprises visual execution data indicative of the desired visual profile to be visual, said visual output unit is configured to execute said visual execution data to obtain the desired visual profile.

22. (canceled)

23. The system of claim 1, wherein at least one of the selected environmental features is a touch-related feature;
wherein said touch-related feature is selected from: texture of different objects in the space, temperature in the space, humidity conditions, elasticity of objects in the space, transparency degree of objects in the space, shape and size of objects in the space, weight of objects in the space, or any combination thereof.

24.-25. (canceled)

26. The system of claim 1, wherein at least one of the selected environmental features is a taste-related feature;
wherein said taste-related feature comprises instructions for types of foods and drinks to be consumed in the space and types of foods and drinks that are to be prevented from consumption in that space.

27. (canceled)

28. The system of claim 1, wherein the physiological response comprises wakefulness, alertness, amazement, concentration, awareness, confusion.

29. The system of claim 1, wherein the physiological response comprises causing a change in the subject in at least one of: certain brain wave activity, hormone secretion, heart rate, body temperature, blood pressure, pupil dilation, respiratory rate or any combination thereof.

30. A method for affecting, planning and/or controlling environmental features in a space for enhancing or inhibiting at least one physiological response of a subject, comprising:
receiving input data of said desired physiological response;
based on the input data, selecting environmental features to be applied on the space, said selected environmental are classified to result in the desired physiological response;
generating a temporal execution profile of said selected environmental features to obtain said physiological response;
outputting an output data indicative of the selected environmental features and the temporal execution profile.

31. The method of claim 30, comprising assigning each environmental feature with a scoring rank for enhancing and/or inhibiting the desired physiological response, said selecting comprises selecting environmental features above a selected threshold of said scoring rank;
wherein said scoring rank is associated with the enhancement or inhibition of a certain brain wave.

32. (canceled)

33. The method of claim 30, wherein the environmental features are clustered to sub-categories, the method comprising selecting one or more environmental features from each sub-category, wherein each sub-category consists environmental features that are recognizable by or affecting a similar sense;
wherein said execution profile comprises a time-dependent execution profile of two or more selected environmental features.

34. (canceled)

35. The method of claim 33, wherein at least one of the selected environmental features is a scent to be dispensed in the space and at least one of the selected environmental features is an acoustic output;
wherein said execution profile comprises N periodical schemes, the execution profile in one periodical scheme comprises similar time-depended relations of executions of said at least two selected environmental features, wherein each two following periodical schemes are separated by a transition scheme that comprises a transition execution profile of at least one environmental feature different than the execution profile in said N periodical schemes;
wherein the scent is selected from synthetic scent, natural scent, or a combination thereof, wherein said temporal execution profile comprises dispensing execution data indicative of scent selection and dispensing profile, the method further comprising dispensing scent into the space based on the dispensing profile.

36.-37. (canceled)

38. The method of claim 33, wherein at least one of the selected environmental features is an acoustic output, wherein said temporal execution profile comprises acoustic execution data indicative of acoustic signal execution profile of said acoustic output, the method further comprising outputting said acoustic execution data to obtain a desired acoustic signal profile;
wherein said acoustic signal profile is composed of:
intensity and/or frequencies profile of the signal;
wherein said acoustic output is composed of two or more acoustic elements selected from: acoustic frequency, soundscape, music, and sub-conscious signals in ASMR technology, wherein each of the two or more acoustic elements is associated with effect on said physiological response, the method further comprising combining two or more acoustic elements to generate said acoustic output to obtain said physiological response; and
comprising generating an acoustic output that comprises acoustic frequencies matching the neural oscillations associated with the desired physiological response.

39.-47. (canceled)
